(19) United States
(12) Patent Application Publication
(10) Pub. No.: US 2007/0031354 A1
(43) Pub. Date: Feb. 8, 2007

(54) Cosmetic and topical compositions comprising cuphea oil and derivatives thereof

(76) Inventors: James H. Brown, Scottsdale, AZ (US); Robert Kleiman, Sun Lakes, AZ (US); John Hill, Mesa, AZ (US); Sambasivarao Koritala, Sun Lakes, AZ (US); Kenneth Lotts, Gilbert, AZ (US)

Correspondence Address: NOBLITT & GILMORE, LLC., 4800 NORTH SCOTTSDALE ROAD, SUITE 6000, SCOTTSDALE, AZ, 85251, US

(21) Appl. No.: 11/196,538

(22) Filed: Aug. 3, 2005

(51) Int. Cl. A61K 8/37; A61K 8/97

(52) U.S. Cl. 424/59; 424/74; 424/70.7; 424/70.31

(57) ABSTRACT

A method for an oxidatively stable cuphea derived emollient composition having a relatively high capric acid concentration is disclosed. Various features and specifications may be controlled, adapted or otherwise modified to improve the application and utilization of cuphea oil and cuphea oil derivatives as emollients. The present invention generally provides cosmetic, personal care and other topical preparation ingredients having improved oxidative stability as well as other desirable characteristics as compared with naturally derived emollient and/or synthetic emollient alternatives.

### FIELD OF INVENTION

The present invention generally concerns topical and cosmetic emollient compositions; and more particularly, representative and exemplary embodiments of the present invention generally relate to the provision of oxidatively stable emollients produced from cuphea oil and its derivatives.

### BACKGROUND

Emollients are materials that are applied topically to the skin of the user to produce softness, smoothness or suppleness. They have been used for centuries in both cosmetic and medicinal products. Historically, emollients consisted of extracts or concentrated materials taken from plants or animals. Modem emollients may additionally include partially synthetic (e.g., derivatives of natural products) or even completely synthetic materials. Natural emollients have generally tended to provide a wet or oily feel and appearance to the skin, whereas synthetic and partially synthetic emollients have been tailored to provide a specific skin-feel and appearance for use in various products. Even with such tailoring, there are relatively few synthetic emollients that are suitably adapted to provide a satisfactory dry feel.

In recent years, there has been an increase in consumer preference for products labeled “all natural”. There has also been increasing interest in the use of natural products obtained from renewable resources—or at least ‘naturally derived’ products. Much of this effort has been directed to the use of naturally occurring, biodegradable materials that require minimal processing. The trend towards the use of natural, biodegradable products in cosmetic preparations has provided manufacturers and compounders with the opportunity for identifying alternatives to synthetic ingredients.

In addition to the skin-feel of an emollient, topical applications and their ingredients must usually exhibit stability both in storage and in use. In general, topical applications must not deteriorate or separate over time and the individual ingredients should not decompose or otherwise undergo chemical changes that alter their otherwise desirable properties. For example, one contributor to ambient damage of formulation ingredients and finished products involves oxidation. Topical cosmetics, fragrances, medicaments, pharmaceutical preparations and colorants that contain natural emollients are generally susceptible to the damaging effects of oxidation.

Conventional means for reducing the effects of oxidation include: oxygen-excluding packaging (e.g., bottles, cans, containers, oxygen impermeable polymer wraps, and/or the like); the chemical modification of ingredients to reduce susceptibility to oxidation while minimally altering otherwise desirable properties; and the direct addition of antioxidants to quench oxidative species before they have the opportunity to oxidize the material of interest.

Packaging controls are generally effective where a product is to be used once; as in the case when a package is opened, air is introduced into the container and the package is configured to provide at least some protection from subsequent persistent contact with oxygen in the atmosphere. Chemical modification of a component ingredient typically offers an improved solution. Of course, chemical alteration assumes that a specific modification may indeed be devised that both substantially reduces the tendency towards oxidation while maintaining the functional properties desired in the selection of the original component ingredients. This may be an exhausting, time-consuming task with no certainty of success.

The use of antioxidants offers a more general solution to the oxidation problem for a wide variety of materials and applications, including the protection of edible foods against premature spoilage. The use of antioxidants might appear to require little more than the selection of a suitable commercially available compound to achieve a viable finished product with the requisite threshold resistance to oxidation; however, antioxidants often produce complexed and unpredictable interactions with other formulation ingredients on a physical and/or chemical level. It is often necessary to conduct extensive research with no assurance of success. Additionally, there are a wide variety of antioxidants and numerous variants that may obstruct a search for a suitable antioxidant additive.

Free-radical terminators are one class of antioxidants. Representative subclasses of free-radical terminators include butylated hydroxyanisole (BHA), butylated hydroxytoluene (BHT), and hydroquinones (such as tertiary-butylhydroquinones, and propyl gallate). Reducing agents or oxygen scavengers embody another class of antioxidants. These include, for example, ascorbic acid (vitamin C) and derivatives thereof (such as esters of ascorbic acid; e.g., ascorbyl palmitate, etc.), sulfites (e.g., alkali metal sulfites and bisulfites, etc.), glucose oxidases (including catalase), erythrobic acid and its derivatives, and the like. Chelating agents comprise yet another class of materials that have been used to address problems with potentiators of oxidation. Representative chelating agents include citric acid (and its derivatives), polyphosphages, and aminopolycarboxylic acids, such as ethylene-diamine-tetra-acetic acid (EDTA). There are also other antioxidant classes which are less generally applicable for use with various topical, pharmaceutical and cosmetic formulations.

With respect to natural and naturally derived oils, recent attention has been directed to cuphea seeds, which are rich in medium chain fatty acids. Lauric acid, for example, is present in generally high concentrations for many species of cuphea. Other species are rich in capric, myristic and other medium-chain fatty acids. In the past, cuphea has not been commercially harvested due to several unfavorable crop traits. These include, for example: intolerance to frost, fragile seed pods which shatter easily, unpredictable flowering, slow germination, and sticky elastic hairs that cover the leaves and flowers. Perhaps the most difficult problem to mitigate has been that of seed shatter, which generally excludes cuphea from conventional harvesting techniques. Recently, however, researchers have addressed this issue by producing interspecific cuphea species that exhibit delayed seed shattering.

Notwithstanding the preceding, there is a present and continuing need for a class of compositions comprising naturally derived oils and waxes: (1) that exhibit extended stability relative to unadulterated oils and waxes alone; (2) that are themselves considered “all natural” and/or organic; (3) that have improved properties as compared with other natural oil and naturally derived materials; and (4) that are more economically obtained.

### SUMMARY OF THE INVENTION

In various representative and exemplary aspects, the present invention discloses the use of cuphea oil having a relatively high level of capric acid in the manufacture of pharmaceutical, cosmetic and topical preparations. Advantages of the present invention will be set forth in the Detailed Description which follows and may be apparent in view of the Detailed Description or may be learned by practice of exemplary embodiments of the invention. Still other advantages of the invention may be realized by means of any of the instrumentalities, methods or combinations particularly pointed out in the claims.

### BRIEF DESCRIPTION OF THE DRAWINGS

Representative elements, operational features, applications and/or advantages of the present invention reside in the details of construction and operation as more fully hereafter depicted, described and claimed—reference being made to the accompanying drawings forming a part hereof, wherein like numerals refer to like parts throughout. Other elements, operational features, applications and/or advantages may become apparent in light of certain exemplary embodiments recited in the Detailed Description, wherein:

FIG. 1 generally illustrates the effect of addition of sunscreen compounds on the oxidative stability of various oils;

FIG. 2 generally illustrates the effect of addition of tocopherols on the oxidative stability of various oils;

FIG. 3 generally illustrates occlusivity values for a variety of natural and naturally derived oils, some in accordance with various representative and exemplary embodiments of the present invention;

FIG. 4 generally illustrates a gas chromatogram of naturally obtained cuphea oil having a relatively high tricaprin concentration;

FIG. 5 generally illustrates a gas chromatogram of catalytically randomized cuphea oil, in accordance with a representative and exemplary embodiment of the present invention;

FIG. 6 generally illustrates a gas chromatogram of catalytically hydrogenated cuphea oil, in accordance with various representative and exemplary embodiments of the present invention; and

FIG. 7 generally illustrates a gas chromatogram of catalytically hydrogenated and randomized cuphea oil, in accordance with various representative and exemplary embodiments of the present invention.

### DETAILED DESCRIPTION OF EXEMPLARY EMBODIMENTS

The following descriptions are of exemplary embodiments of the invention and the inventors' conception of the best mode and are not intended to limit the scope, applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Cuphea, of the Lythraceae family, contains over 260 species of plants that grow in temperate and subtropical regions around the world. Most cuphea seeds are rich in medium chain fatty acids. Lauric acid, for example, is present in relatively high concentrations. Certain interspecific crosses of cuphea, such as C. lancolata and C. viscosissoma, produce relatively high percentages of other medium chain fatty acids as well; specifically 60-75% capric acid by concentration.

Cuphea oil has not been commercially harvested due to several unsatisfactory crop traits, such as, for example: intolerance to frost, fragile seed pods which tend to shatter easily, indeterminate flowering, slow germination, and sticky elastic hairs that cover the leaves and flowers. Perhaps the most difficult problem has been that of seed shatter, which previously excluded cuphea from conventional harvesting methods. Recently however, researchers have addressed the seed shatter problem by producing interspecific cuphea plants that delay or even completely halt seed shattering.

In recent years, there has been an increase in consumer interest for products labeled “all natural”. There has also been a desire for using natural products from renewable resources—or at least naturally derived products. Much of this effort has been directed to employing natural, biodegradable materials that require relatively little pre- and/or post-processing. The trend towards the use of “all natural” products in cosmetic preparations has provided manufacturers and compounders with the opportunity and economic incentive to identify alternatives to synthetic ingredients.

Natural emollients generally tend to provide a wet or oily feel and appearance to the skin, while synthetic and partially synthetic emollients have been customized inter alia to provide a drier skin-feel and/or appearance. Even so, there are relatively few synthetic compositions that provide a satisfactory dry feel. Additionally, various regulatory bodies may restrict the use of the phrase “all natural” as applied to products that contain synthetic ingredients.

In addition to the feel of an emollient upon application to the skin, topical preparations and their ingredients must generally exhibit oxidative stability, both in storage and in use. Many topical applications such as cosmetics, fragrances, medicaments, and colorants that contain natural emollients are known to exhibit susceptibility to the damaging effects of oxidation.

The present invention discloses representative and exemplary compositions that are useful as emollients in topical applications. One particular class of exemplary compounds include cuphea derived fatty acid esters having the general formula:
R₁—COO—R₂
where R₁ corresponds to (CH₂)ₙCH₃ where 0≦n ≦17 (including isomers thereof) and R₂ corresponds to an aliphatic residue comprising (CH₂)ₓCH₃ where 0≦x≦13.

Interesterification and esterification reactions of cuphea and cuphea derived fatty acids were conducted at elevated temperatures (e.g., 80-150° C.) with an alkaline catalyst—e.g., 0.5 to 1% sodium methoxide (sodium methylate). It will be appreciated that various other catalysts may be employed to produce a substantially similar result. Accordingly, any catalyst, whether now known, hereafter derived or otherwise described in the art, may be alternatively, conjunctively or sequentially employed. For example, representative catalysts that may be used include sodium hydroxide, para-toluene sulfonic acid and/or the like.

Additional representative cupheate derived compositions, in accordance with various exemplary embodiments of the present invention, may also include other compounds, such as salts of cuphea derived fatty acids. Representative fatty acid components of cuphea and cuphea derived compounds, in accordance with various embodiments of the present invention, may include, for example: 6:0 (caproic); 8:0 (caprylic); 10:0 (capric); 12:0 (lauric); 14:0 (myristic); 16:0 (palmitic); 16:1 (palmitoleic); and/or the like.

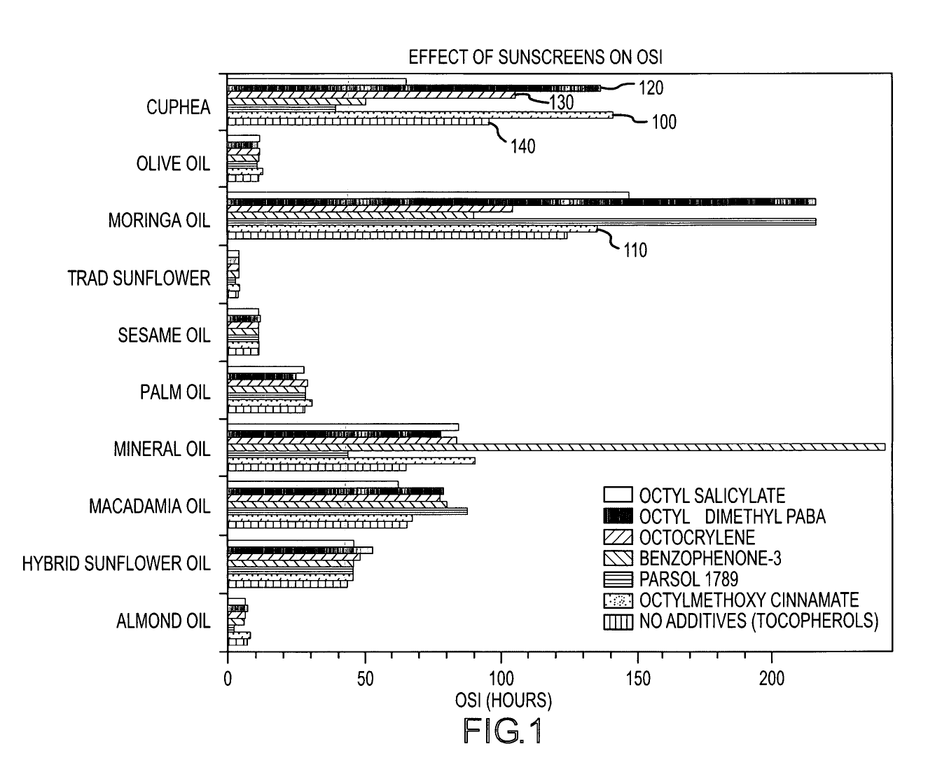

As generally depicted in FIG. 1, when cuphea oil having a concentration of at least 40% capric acid is added to various sunscreen compounds (e.g., octyl salicylate, octyl dimethyl PABA, octocrylene, benzophenone-3, Parsol 1789, and octylmethoxy cinnamate), the oxidative stability of the resulting sunscreen preparations are increased relative to nearly all other oil formulations (e.g., olive, traditional sunflower, sesame, palm kernel, mineral, macadamia, hybrid sunflower, and almond); with several examples corresponding to moringa oil providing a notable exception, as well as that of mineral oil with benzophenone-3.

FIG. 1 representatively illustrates:

with the addition of no other additives other than tocopherols, cuphea 140 is exceeded only by moringa oil in its oxidative stability;

with the addition of octylmethoxy cinnamate to cuphea 100, the oxidative stability of the resulting formulation exceeds all other cinnamate mixtures, including that of moringa 110;

with the addition of PABA to cuphea 120, the oxidative stability of the resulting formulation exceeds all other PABA mixtures, again with the exception of moringa;

and

with the addition of octocrylene to cuphea 130, the oxidative stability exceeds all other octocrylene formulations, and at least substantially matches the oxidative stability of moringa.

Accordingly, it can be seen that the oxidative stabilities of the disclosed sunscreen compounds are generally improved with the addition of cuphea oil relative to the majority of other oils in the majority of the formulation examples, with the notable exception, of course, of moringa. Consequently, sunscreens, sun blocks and ultraviolet absorbers admixed with high capric acid concentration cuphea oil may be stored and used for longer periods of time as compared with similar sunscreens, sun blocks or ultraviolet absorbers admixed with other emollients.

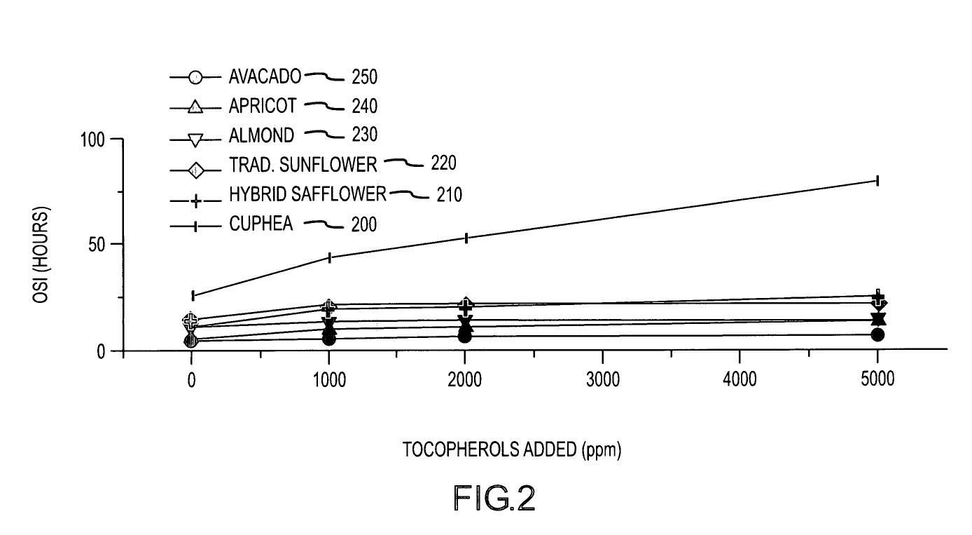

FIG. 2 generally depicts the effect of increasing concentration of tocopherols (from 0 ppm to 5000 ppm) on the oxidative stability of various oils (e.g., avocado 250, apricot 240, almond 230, traditional sunflower 220, hybrid safflower 210, and cuphea 200). As shown in FIG. 2, cuphea 200 demonstrates a significant increase in oxidative stability with the addition of tocopherols relative to the disclosed natural oil alternatives (210, 220, 230, 240 and 250).

In another representative application, in accordance with an exemplary embodiment of the present invention, the disclosed compositions generally comprise cuphea oil and cuphea derived compounds with a relatively high tricaprin concentration (e.g., at least about 40%) in contrast to naturally obtained cuphea oils having high lauric acid concentration. Such compositions may be formulated with or used as a substitute for other medium chain triglycerides, such as palm kernel oil, coconut oil, and babassu oil. Additionally, such compositions may also be formulated with silicon-based compounds, synthetic emollients, mineral oil, cocoa butter, shea butter, olive oil, safflower oil, almond oil, apricot oil, sunflower oil, vegetable oil, as well as any other plant oil, whether now known or hereafter described in the art.

The disclosed compositions may be used in cosmetics, foundations, mascaras, leave-in conditioners, massage oils, organic and inorganic pigments, lotions, topical medicaments, ultraviolet radiation absorbers, sunscreens, suntan lotions, sun tan oils, repellants, creams, ointments, powders, soaps, fragrances, scrubs, cleansers, waxes, gels, detergents, sanitizers, balms, glosses, lip sticks, lip glosses, lip balms, cosmetic removers, and/or the like. It will be appreciated that the disclosed cupheate compounds, in accordance with representative and exemplary embodiments of the present invention, may be compounded with various other ingredients to produce any type of cosmetic, personal care, topical preparation or pharmaceutical medicament, whether now known or otherwise hereafter described in the art.

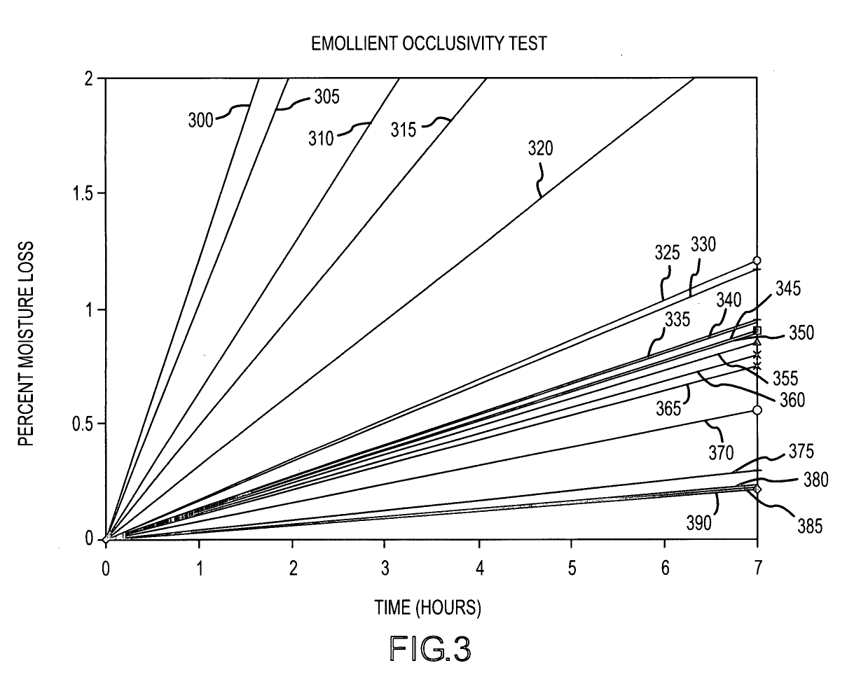

Compositions in accordance with representative embodiments of the present invention have several advantages over other emollients. As generally depicted in FIG. 3 for example, the occlusivity of cupheate derived compounds (e.g., cupheanyl acetate 310, ethyl cupheate 315, and cuphea alcohols 320) are substantially enhanced or otherwise modified as compared with that of naturally obtained cuphea 340, or other emollients such as soybean oil 345, castor oil 350, macadamia oil 355, jojoba oil 360, moringa oil 365, mineral oil 370, Moringa Esters 30 (375), Moringa Esters 60 (380), Moringa Esters 75 (385), and petrolatum 390. A substantially lower occlusivity generally imparts a dryer skin-feel when applied topically, as compared with the greasy feeling usually attributed to compounds having higher water occlusivity. This is due in part to higher transpirational water permeability of cuphea and cuphea derived emollient compounds.

An added benefit of representative compositions in accordance with various exemplary embodiments of the present invention is that they generally provide a range of low occlusivity values, as seen for example in FIG. 3. Specifically, the cuphea derived compositions in accordance with the present invention where R₁ comprises methyl 310 (i.e., n=0) or ethyl 315 (i.e., n=1) generally provide a lower occlusivity value than in the case where R₁ comprises larger alkyl residues, such as decyl 330 (i.e., n=9). Accordingly, it will be appreciated that the present invention further provides a mechanism for controlling or otherwise modifying the transpirational occlusivity of the disclosed compositions of the present invention in order to obtain a desired skin-feel.

Representatively disclosed cuphea derived compositions, in accordance with exemplary embodiments of the present invention, also demonstrate the lowest slip value (e.g., 71°) of any emollient ever observed by the Applicants. Many users of topical preparations prefer not to have the feeling of “slipperiness”. A specific example of this may include athletes whose performance may be adversely impacted, for example, by using a sunscreen or sun block comprising an emollient having a characteristically higher slip value. Thus, having an emollient with a low slip value would be beneficial in certain applications and may have specific commercial utility in topical preparations such as those of sunscreens or sun blocks.

Representatively disclosed cuphea derived compositions, in accordance with exemplary embodiments of the present invention, also exhibit larger spread values as a function of time as compared with naturally obtained cuphea oil. This may be beneficial in applications involving topical preparations that are adapted to provide greater material coverage with less mechanical spreading or rubbing. Representatively disclosed cuphea derived compositions, in accordance with exemplary embodiments of the present invention, also demonstrate a lower refractive index than that of naturally obtained cuphea oil.

Some of the representatively described characteristics and benefits of the disclosed cuphea derived compositions, in accordance with various exemplary embodiments of the present invention, may at least be partially attributable to relatively high volatility of the derived compounds in terms of their vapor pressure. It has been suggested that such characteristically high volatility may give rise, at least in part, to the observed values for slip, occlusivity and spread. It will be appreciated that various other characteristics may be enhanced or otherwise improved with the utilization of cuphea derived emollient formulations that demonstrate relatively high volatility, whether now known or otherwise hereafter described in the art.

A representative and exemplary method for making a cuphea derived composition, in accordance with another embodiment of the present invention, generally includes the steps of providing cuphea oil having a capric acid concentration of at least about 40% and reacting the cuphea oil with a randomization catalyst over heat.

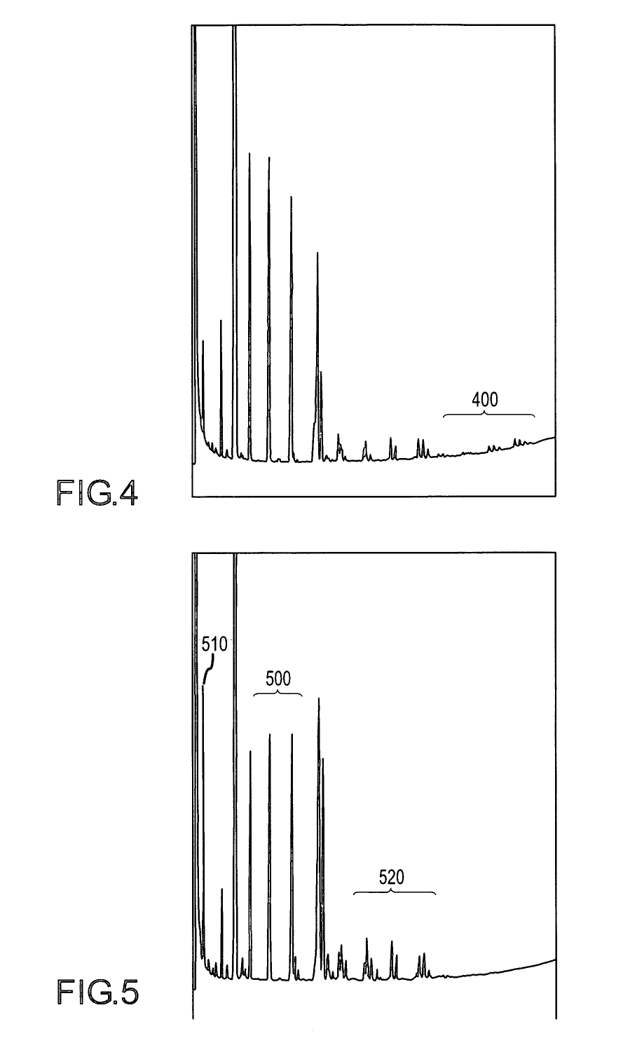

FIG. 4 depicts a gas chromatogram taken for naturally obtained cuphea oil having a relatively high tricaprin concentration. FIG. 5 illustrates a gas chromatogram taken of catalytically randomized cuphea oil, in accordance with a representative and exemplary embodiment of the present invention. As representatively depicted in these chromatograms, the catalytically randomized product mix generally has fewer high molecular weight triglycerides (see FIG. 5) as compared with those appearing in the distribution of cuphea oil starting material 400. Some low molecular weight peaks in the randomized product mix (e.g., 510) are substantially increased, while some mid-range molecular weight components (e.g., 500) are somewhat decreased. It is important to here note that the vertical and horizontal scales of FIG. 4 and FIG. 5 are substantially identical.

The randomized mix exhibits a substantially lower melting point, as compared with naturally obtained cuphea oil, as well as a lower and dramatically unexpected cloud point. The substantially low precipitate content is quite surprising as compared with other oils (e.g., moringa oil), which generally tend to have higher precipitate content after randomization. Both a low melting point and low precipitate content generally provide favorable benefits in terms of thermal stability as well as various other physical characteristics related to product formulation.

The randomized cuphea product mix also demonstrates a lower viscosity (as measured at 40° C.) than that of the natural oil starting material. A lower viscosity generally imparts a thinner feel when topically applied, as compared with the thick feel typically associated with most oils and oil derived products.

An exemplary method for making a cuphea derived composition, in accordance with another representative embodiment of the present invention, generally includes the steps of:

reacting hydrogen gas with cuphea oil having a relatively high capric acid concentration in the presence of a catalyst, such as heterogeneous metal or nickel, palladium, ruthenium, platinum or rhodium-based catalysts up to about 20° C. to about more than 200° C. with gas pressures ranging from up to about atmospheric pressure to about more than 200 psi;

interesterifying the resulting hydrogenated product with sodium methylate (or other suitably adapted randomization catalyst) over heat; and

isolating the resulting randomized triglyceride mixture.

The resulting hydrogenated and randomized cuphea derived product mix generally comprises 30-35% tricaprin.

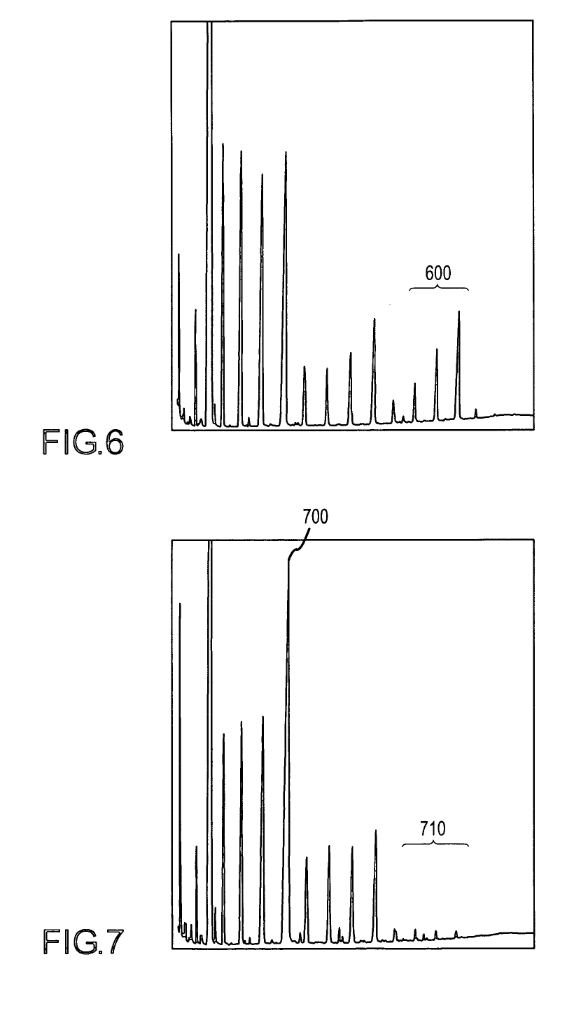

FIG. 6 depicts a gas chromatogram of catalytically hydrogenated cuphea oil, in accordance with various representative and exemplary embodiments of the present invention. FIG. 7 generally illustrates a gas chromatogram of catalytically hydrogenated and randomized cuphea oil, in accordance with various representative and exemplary embodiments of the present invention.

As representatively depicted in the chromatograms of FIGS. 6 and 7, the hydrogenated and randomized product mixture (FIG. 7) generally has fewer high molecular weight triglycerides 710 than those appearing in the distribution of hydrogenated cuphea 600, while certain mid-range molecular weight species of hydrogenated and randomized product 700 are substantially increased. It is important to note that the vertical and horizontal scales of FIG. 6 and FIG. 7 are substantially identical.

The melting point of the hydrogenated and randomized product mixture is substantially higher than the pre-randomized product, such that the hydrogenated and randomized product is solid at room temperature. Also, the cloud point and viscosity are substantially higher than that of the pre-randomized product.

In the foregoing specification, the invention has been described with reference to specific exemplary embodiments; however, it will be appreciated that various modifications and changes may be made without departing from the scope of the present invention as set forth in the claims below. The specification and Figures are to be regarded in an illustrative manner, rather than a restrictive one and all such modifications are intended to be included within the scope of the present invention. Accordingly, the scope of the invention should be determined by the claims appended hereto and their legal equivalents rather than by merely the examples described above. For example, the steps recited in any method or process claims may be executed in any order and are not limited to the specific order presented in the claims. Additionally, the components and/or elements recited in any composition claims may be aggregated in a variety of permutations to produce substantially the same result as the present invention and are accordingly not limited to the specific aggregation recited in the claims.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features of components of any or all the claims. As used herein, the terms “comprises”, “comprising”, “having”, “including” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

What is claimed is:

1. A composition substantially derived from cuphea oil for topical application to the skin, said composition comprising:
at least one cupheate ester corresponding to the formula:
R_{1—COO—R}₂
wherein R₁ is selected from the group consisting of (CH₂)ₙCH₃ where 0≦n≦17, including isomers thereof, and
R₂ comprises an aliphatic residue corresponding to (CH₂)ₓCH₃ where 0≦x≦13.

2. The composition of claim 1, further comprising a substantially high concentration of R₂ where x=1.

3. The composition of claim 1, further comprising a substantially high concentration of aliphatic residue where n=9.

4. The composition of claim 3, wherein the occlusivity of said composition is substantially enhanced relative to naturally obtained cuphea oil.

5. The composition of claim 4, wherein enhancement corresponds to at least one of relative increase and relative decrease in occlusivity.

6. The composition of claim 1, wherein the refractive index of said composition is at least lower than that of naturally obtained cuphea oil.

7. The composition of claim 1, wherein the slip of said composition is at least lower than that of naturally obtained cuphea oil.

8. The composition of claim 1, where the viscosity of said composition is at least lower than that of naturally obtained cuphea oil.

9. The composition of claim 1, where the spread as a function of time of said composition is at least higher than that of naturally obtained cuphea oil.

10. The composition of claim 1, wherein said cuphea derived composition is substituted for at least one of a silicon-based compound, a synthetic emollient, a medium chain triglyceride, mineral oil, coconut oil, palm kernel oil, babassu oil, cocoa butter, shea butter, olive oil, safflower oil, almond oil, apricot oil, sunflower oil, vegetable oil, and a plant oil.

11. The composition of claim 1, wherein said cuphea derived composition further comprises a component ingredient in at least one of a cosmetic, a personal care item, a foundation, a mascara, a leave-in conditioner, an eye shadow, an eyeliner, a lip liner, a lip stick, a lip balm, a massage oil, an inorganic pigment, a organic pigment, a lotion, a topical medicament, an ultraviolet radiation absorber, a sunscreen, a suntan lotion, a sun tan oil, a repellant, a cream, an ointment, a powder, a soap, a fragrance, a scrub, a cleanser, a wax, a gel, a detergent, a sanitizer, a balm, a gloss, and a cosmetic remover.

12. The composition o claim 1, wherein said cuphea derived composition is at least partially substituted for a substantially synthetic emollient compound.

13. A composition wherein the oxidative stability of at least one of a sunscreen, a sun block, and an ultraviolet absorber is at least one of enhanced and not substantially degraded with addition of cuphea oil having a capric acid concentration of at least 40%.

14. The composition of claim 13, further comprising at least one of tocopherol octylmethoxy cinnamate, octocrylene, and octyl dimethyl PABA.

15. The composition of claim 13, wherein said composition is at least partially substituted for a substantially synthetic emollient compound.

16. A method of randomizing cuphea oil, said method comprising the steps of: providing cuphea oil; and
reacting said cuphea oil with a randomization catalyst over heat.

17. The method of claim 16, wherein said randomization catalyst comprises at least one of sodium methylate, sodium hydroxide, and para-toluene sulfonic acid.

18. The method of claim 16, wherein the resulting product has a substantially lower tricaprin concentration as compared with that of the pre-randomized cuphea starting material.

19. The method of claim 16, wherein the resulting product has a substantially lower melting point than that of the pre-randomized cuphea starting material.

20. The method of claim 16, wherein the resulting product has a substantially lower cloud point than that of the pre-randomized cuphea starting material.

21. The method of claim 16, wherein the resulting product has a lower viscosity than that of the pre-randomized cuphea oil starting material.

22. The method of claim 16, wherein the resulting product is used to at least partially substitute for a synthetic emollient.

23. A method of hydrogenating and randomizing cuphea oil, said cuphea oil having a capric acid concentration of at least 40%, said method comprising the steps of:
reacting hydrogen gas with said cuphea oil between about 20° C. to at least about 200° C., between about atmospheric pressure to at least about 200 psi in the presence of a hydrogenation catalyst; and
introducing said hydrogenated fatty acid mixture to a randomization catalyst at elevated temperature.

24. The method of claim 23, wherein said hydrogenation catalyst comprises at least one of a heterogeneous metal catalyst, nickel, palladium, ruthenium, platinum and rhodium-based catalyst.

25. The method of claim 23, wherein said randomization catalyst comprises at least one of sodium methylate, sodium hydroxide, and para-toluene sulfonic acid.

26. The method of claim 23, wherein the resulting product has a substantially lower tricaprin concentration as compared with that of the pre-randomized, pre-hydrogenated cuphea oil starting material.

27. The method of claim 23, wherein the resulting product has an at least lower viscosity as compared with that of the pre-randomized cuphea oil starting material.

28. The method of claim 23, wherein the resulting product mix has an at least partially smaller content of higher molecular weight triglycerides as compared with that of the pre-randomized cuphea oil starting material.

29. The method of claim 23, wherein the resulting product mix has an at least higher spread value than that of the pre-randomized cuphea oil starting material.

30. The method of claim 23, wherein the resulting product mix has an at least lower occlusivity that that of the pre-randomized cuphea oil starting material.

31. The method of claim 23, wherein the resulting product mix has an at least lower slip than that of the pre-randomized cuphea oil starting material.

32. The method of claim 23, wherein the resulting product mix is used to at least partially substitute for a synthetic emollient.

33. The use of cuphea oil having a capric acid concentration of at least 40% in the manufacture of at least one of a pharmaceutical, a cosmetic, a personal care item and a topical preparation.
